(19) United States
(12) Patent Application Publication
(10) Pub. No.: US 2024/0420461 A1
(43) Pub. Date: Dec. 19, 2024

(54) CONTEXTUALIZING THE ANALYSIS OF MEDICAL IMAGES

(71) Applicant: KONINKLIJKE PHILIPS N.V., EINDHOVEN (NL)

(72) Inventors: Axel SAALBACH, HAMBURG (DE); Nicole SCHADEWALDT, NORDERSTEDT (NL); Steffen RENISCH, HAMBURG (DE); Heinrich SCHULZ, HAMBURG (DE); Matthias LENGA, MAINZ (DE)

(21) Appl. No.: 18/703,735

(22) PCT Filed: Sep. 28, 2022
(86) PCT No.: PCT/EP2022/076880

§ 371 Date: Apr. 23, 2024

Related U.S. Application Data
(60) Provisional application No. 63/272,259, filed on Oct. 27, 2021.

(51) Int. Cl. G06V 10/82; G06V 10/25; G06V 10/44; G06V 10/77; G06V 10/80; G16H 30/40; G16H 50/20

(52) U.S. Cl. CPC G06V 10/82; G06V 10/25; G06V 10/44; G06V 10/7715; G06V 10/806; G16H 30/40; G16H 50/20; G06V 2201/03

(57) ABSTRACT

A mechanism for providing additional information about a medical image analysis process. A neural network is used to process a medical image, and non-image data elements, to generate an output. An influence of different regions of the medical image and of the non-image data elements to an output of the neural network or intermediate layer output (of the neural network) is determined.

### FIELD OF THE INVENTION

The present invention relates to the field of medical image analysis, and in particular to the field of analysis of medical images.

### BACKGROUND OF THE INVENTION

There is an increasing interest in the use of Deep Learning (DL) techniques across a range of applications in the medical domain. One particular area of interest is in the analysis of medical image, such as in the identification or classification of one or more target findings of the medical image. Example findings include the predicted presence or absence of a particular structural element, abnormality, disease, condition, pathology or status of the anatomy represented by the medical image. By way of example, a deep learning technique may be configured to predict the presence or absence of pneumothorax (or other condition/disease/pathologies) in a chest X-ray image.

The success rate of modern deep learning techniques (e.g. in performing a classification task) has been empirically shown to be on par or even superior to those human experts. However, at the same time, the limited interpretability and understandability of deep learning models remains a point of concern, and reducing overall the acceptance of DL for medical applications.

In order to overcome these limitations, methods for model interpretation have been developed that allow for the visualization of image regions relevant for the analysis or classification process.

For instance, Ivo M. Baltruschat, Hannes Nickisch, Michael Grass, Tobias Knopp, Axel Saalbach, Comparison of Deep Learning Approaches for Multi-Label Chest X-Ray Classification, Nature Scientific Reports demonstrates that such techniques allow for the localization of pathologies (e.g. a pneumothorax), thus providing insights into the deep learning techniques decision path.

Moreover, providing a clinician with information that identifies the key area(s) (of the medical image) that resulted in the particular output provides a clinician with valuable information usable in their assessment of the imaged subject. In particular, this indicates important areas that may warrant closer attention by the clinician and/or areas that may indicate the presence of foreign objects that may have disrupted the analysis process.

There is an ongoing desire to improve the information made available to a clinician.

### SUMMARY OF THE INVENTION

The invention is defined by the claims.

According to examples in accordance with an aspect of the invention, there is provided a computer-implemented method of determining the influence of inputs to a neural network, that processes medical data, on an output of the neural network. The computer-implemented method comprises: defining a neural network configured to process medical data to produce the output, the medical data comprising a medical image, comprising a plurality of different regions, and one or more non-image data elements, wherein the neural network is configured to: partially process the medical image using an image processing branch to derive one or more image features; combine the image features with the one or more non-image data elements to form intermediate inputs for a combined processing branch of the neural network; and process the intermediate inputs using the combined processing branch of the neural network to generate the output of the neural network; calculating, for each of region of the medical image, a numeric value representing the influence of the region on the output of neural network; calculating, for each non-image data element, a numeric value representing the influence of the data element on the output of the neural network; and determining, for each region of the medical image and each non-image data element, an indicator of the calculated numeric value representing the influence of the region of the medical image or the non-image data element.

The present disclosure proposes an approach for generating indicators representing the influence of different inputs to an output of the neural network. This provides an approach for attributing the cause of the (content of the) output of the neural network to one or more regions of the medical image and/or one or more non-image data elements.

Embodiments propose to effectively quantify the influence of different regions and/or non-image data elements on the output of the neural network. This quantified value is then used to define an indicator that effectively indicates whether or not said region or data element has an influence on the output of the neural network.

Embodiments thereby provide provides valuable information for assessing the main cause(s) of the output of the neural network. This information effectively highlights areas that would benefit from further investigation or attention from a clinician or operator. In particular, the information can be used to assess a largest contributory cause of an output of the neural network, which can aid in the determination of an appropriate treatment for a subject (e.g. by identifying a cause that could be resolved or addressed to treat an identified pathology) or to improve a diagnosis of a subject (e.g. by facilitating identification of a cause known to provide an incorrect output of the neural network).

Embodiments thereby provide additional information for aiding a clinician in making a clinical decision, and present embodiments thereby act as a clinical aid. Of course, indicators generated by proposed embodiments find further use in additional processing methodologies.

The method may comprise a step of providing, at a user interface, a user-perceptible output responsive to each generated indicator.

Each indicator may have a numeric indicator having a value equal to the calculated numeric value.

In some examples, the step of calculating, for each non-image data element, a numeric value comprises processing weights of the neural network to produce the numeric value representing the influence of the non-image data element.

In some examples, the combined processing branch comprises a fully connected layer that receives the intermediate inputs and an activation function that processes the output of the fully connected layer to generate the output of the neural network.

Optionally, the calculating, for each of region of the medical image, a numeric value comprises calculating a numeric value that represents the influence of the region on the output of the fully connected layer; and/or the calculating, for each non-image data element, a numeric value comprises calculating a numeric value that represents of the data element on the output of the neural network.

The step of calculating, for each non-image data element, a numeric value may comprise, for each non-image data element: identifying a weight applied to the non-image data element by the fully-connected layer of the neural network in producing the output of the fully-connected layer; and calculating the product of the identified weight and the value of the non-image data element as the numeric value.

In some examples, the image processing branch comprises a penultimate layer that produces one or more feature maps, wherein regions of each feature map correspond to regions of the medical image, and a final layer that produces an image feature from each feature map; and calculating, for each of region of the medical image, a numeric value comprises: for each image feature: identifying a weight applied to the image feature by the fully-connected layer to produce the output of the fully-connected layer; and determining the product of the identified weight and the feature map, from which the image feature is derived, to produce a weighted feature map, and determining, for each region of the medical image, a numeric value representing the influence of the region of the medical image based on the weighted feature maps.

The regions of the feature map may map or correspond to different regions of the medical image. Thus, each region of the feature map may represent a different area (e.g. pixel or group of pixels) of the medical image. The positional and spatial relationship between regions of the feature map and the regions of the medical image can be determined in advance.

The penultimate layer may be a convolutional layer and the final layer may be a pooling layer, e.g. a max pooling layer or an average pooling layer.

In at least one embodiment, the step of determining, for each region of the medical image, a numeric value comprises: summing the weighted features maps to produce a class activation map, the class activation map contains numeric values representing the influence of different regions of the medical image on the output of the neural network.

In some embodiments, the step of calculating, for each non-image data element, a numeric value representing the influence of the data element on the output of the neural network comprises using a gradient class activation mapping technique.

In at least one embodiment, each non-image data element is a respective value and the step of calculating, for each non-image data element, a numeric value comprises, for each non-image data element: computing the gradient of the output with regard to the non-image data element; and calculating the product of the computed gradient and the non-image data element as the numeric value.

This approach makes use of a gradient class activation mapping (CAM) approach to assess the influence of the non-image data elements. Of course, the skilled person would be make use of a gradient class activation mapping approach for assessing the influence of each region of the medical image on the output of the neural network.

In some embodiments, the step of calculating, for each non-image data element, a numeric value representing the influence of the data element on the output of the neural network comprises using an ablation class activation mapping technique.

For example, in some embodiments, the output of the neural network and each non-image data element is a respective value and the step of calculating, for each non-image data element, a numeric value comprises, for each non-image data element: obtaining a first value of the output of the neural network when the non-image data element is omitted from first medical data input to the neural network; obtaining a second value of the output of the neural network when the non-image data element is included in second medical data input to the neural network, the second medical data being otherwise identical to the first medical data; defining a difference between the first value and the second value as a difference value; and calculating the product of the difference value and the non-image data element as the numeric value.

This approach makes use of an ablation class activation mapping approach to assess the influence of the non-image data elements. Of course, the skilled person would similarly be able to make use of an ablation class activation mapping approach for assessing the influence of each region of the medical image on the output of the neural network.

Each indicator may be a binary indicator that indicates whether or not the numeric value exceeds a predetermined threshold.

In some examples, the method comprises providing, at a display, a visual representation of each non-image data element; and visually emphasizing the visual representations of any non-image data element associated with a binary value that indicates that the numeric value for the non-image data element exceeds the predetermined threshold.

In some examples, the method comprises providing, at a display, a visual representation of the medical image; and visually emphasizing the visual representations of any region of the medical image associated with a binary value that indicates that the numeric value for the region of the medical image exceeds the predetermined threshold.

There is also proposed a computer program product comprising computer program code means which, when executed on a computing device having a processing system, cause the processing system to perform all of the steps of any herein described method.

There is also proposed a processing system for determining the influence of inputs to a neural network on an output of the neural network generated by the neural network.

The processing system is configured to: define a neural network configured to process medical data to produce the output, the medical data comprising a medical image, comprising a plurality of different regions, and one or more non-image data elements, wherein the neural network is configured to: partially process the medical image using an image processing branch to derive one or more image features; combine the image features with the one or more non-image data elements to form intermediate inputs for a combined processing branch of the neural network; and process the intermediate inputs using the combined processing branch of the neural network to generate the output of the neural network; calculate, for each of region of the medical image, a numeric value representing the influence of the region on the output of the neural network; calculate, for each non-image data element, a numeric value representing the influence of the data element on the output of the neural network; and determine, for each region of the medical image and each non-image data element, an indicator of the calculated numeric value representing the influence of the region of the medical image or the non-image data element.

The neural network may comprise any herein described neural network.

The processing system may be further configured to use the neural network to process the medical image and the one or more non-image data elements to generate the output of the neural network.

There is also proposed a system comprising: any herein described processing system; and a user interface configured to provide a user-perceptible output responsive to each generated indicator.

There is also proposed a system that comprises any herein described processing system; and a medical imaging device configured to generate the medical image. The system may further comprise a user interface configured to provide a user-perceptible output responsive to each generated indicator.

There is also proposed a system that comprises any herein described processing system; and a memory unit configured to generate the medical image and/or the non-image data elements. The system may further comprise a user interface configured to provide a user-perceptible output responsive to each generated indicator. The system may comprise a medical imaging device configured to generate the medical image.

Any herein described processing system may be appropriately configured to perform any herein described method, and vice versa.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a neural network for use in an embodiment;

FIG. 2 illustrates another neural network for use in an embodiment;

FIG. 3 illustrates a method according to an embodiment;

FIG. 4 illustrates a visual representation provided by an embodiment;

FIG. 5 illustrates a processing system according to an embodiment; and

FIG. 6 illustrates a system according to an embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a mechanism for providing additional information about a medical image analysis process. A neural network is used to process a medical image, and non-image data elements, to generate an output (e.g. a classification or score). An influence of different regions of the medical image and of the non-image data elements to the output (of the neural network) is determined.

The present invention relies on an underlying recognition that valuable information for aiding in the assessment of a medical image (e.g. by a clinician or caregiver) can be identified through determining the features or regions (of the medical image) that have the largest influence the output of a neural network.

Embodiments may be employed in any suitable medical image analysis process, such as those use in the automated analysis and/or diagnosis of chest X-ray data. In particular, embodiments may be employed in any suitable medical image classification, analysis and/or scoring method that makes use of a neural network.

In the context of the present disclosure, a subject may be a human or an animal under the care and/or responsibility of a clinician. The term “patient” may be interchangeable with the term “subject”.

In the context of the present disclosure, a medical image is any image of a subject or patient that has been generated for the purposes of clinical assessment or analysis. Known medical imaging modalities include: X-ray imaging, CT imaging (a form of X-ray imaging), MR imaging, PET imaging, ultrasound imaging and so on. A medical image may be generated using any one or more of these imaging modalities.

The invention proposes to overcome the problems pertaining to analysis of the methodology used by image-analysis deep-learning methods that make use of image and non-image data by extending usage of attribution techniques, such as (e.g. classical) class activation mapping (CAM), to non-image data. The proposed approach also be used for other attribution techniques such as Integrated Gradients, Ablation CAM and so on. An attribution technical identifies the influence of different data portions to the analysis performing by a deep learning method. This approach allows for highlighting or identifying of both image regions and non-image data used by the DL method in its determination.

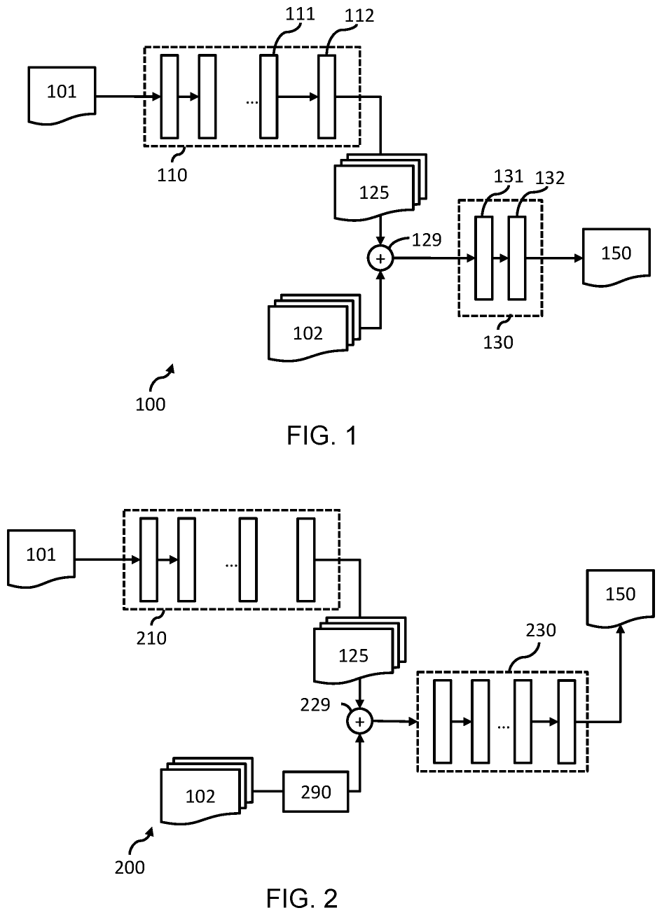

FIG. 1 illustrates an exemplary neural network 100 for use with embodiments.

The neural network 100 is configured to process medical data, including a medical image 101 and non-image data elements 102, to generate an output result, such as a classification or score. Use of non-image data when processing the medical image provides additional context and/or features for improved accuracy in generating the output.

The structure of an artificial neural network (or, simply, neural network) is inspired by the human brain. Neural networks are comprised of layers, each layer comprising a plurality of neurons. Each neuron comprises a mathematical operation. In particular, each neuron may comprise a different weighted combination of a single type of transformation (e.g. the same type of transformation, sigmoid etc., but with different weightings). In the process of processing input data, the mathematical operation of each neuron is performed on the input data to produce a numerical output, and the outputs of each layer in the neural network are fed into the next layer sequentially. The final layer provides the output.

Methods of training a machine-learning algorithm are well known. For the sake of completeness, an example of how to appropriately train a neural network is provided later in this disclosure.

The medical image 101 is processed using an image processing branch 110 to generate one or more image features 125. The image processing branch may process the medical image 101 using one or more pooling and/or convolutional layers, although other forms of layers suitable for performing image processing using a neural network architecture will be apparent to the skilled person.

In some example, the image processing branch 110 comprises a penultimate (i.e. second to last) layer 111 that generates one or more feature maps. The penultimate layer 111 may be a convolutional layer. Each feature map is a feature-domain representation of the medical image 101, (and may be of the same resolution or different resolution to the medical image 101. Different regions of the feature map correspond to different regions of the medical image. For instance, an upper left quadrant of the feature map may correspond to an upper left quadrant of the medical image.

The smallest addressable unit of the feature map may thereby correspond to a pixel or group of pixels/voxels of the medical image. The relationship between the smallest addressable unit of the feature map and the medical image can be established in advance, based on the particular structure of the neural network.

The image processing branch 110 may also comprise a final layer 112 that produces an image feature from each feature map. The final layer 102 may, for instance, be a pooling layer (such as a max pooling or average pooling) layer that produces (for each feature map) an image feature representing the overall feature map.

Other example outputs and configurations for an image processing branch will be apparent to the skilled person, based on alternative approaches for processing medical images to produce image features 125.

The image features 125 are combined with non-image data elements 102, which are derived from medical non-image data. For instance, the non-image data elements may contain a data representation of medical non-image data.

Examples of non-image data elements include features responsive to information and/or characteristics of a subject being imaged, the imaging device and/or the operator of the imaging device. Examples include, for instance, an age of the subject, a gender of the subject, medical history of the subject, a type of imaging being performed, an experience, role and/or seniority of the operator of the imaging device and so on.

Combining the image features 125 with non-image data elements 102 is performed by a combining process 129. The process 129 may, for instance, concatenate the image features with the non-image data elements. In some instances, the process 129 may comprise concatenating metadata in an image/feature map like representation to the image features 125.

The combination of the features forms intermediate inputs, which are then fed as input to a combined processing branch 130 of the neural network 100. The combined processing branch processes the combination of the image features 125 and the non-image data elements 102 to produce an output of the neural network.

In the illustrated embodiment, which provides one working example, the combined processing branch 130 comprises a fully connected layer 131 and an activation function 132. Thus, the image and non-image data elements form inputs to the fully connected layer. The output of the fully connected layer is processed using the activation function 132 to generate a classification result (which here forms the output 150 of the neural network). The activation function 132 may, for instance, be a sigmoid function, such as a softmax function or (Heaviside/binary) step function.

In the illustrated example, the neural network is configured to generate a single classification result, e.g. the neural network is designed for a single classification process. However, the neural network 100 may easily be reconfigured or designed for a multi-classification process (to produce multiple classification results).

Similarly, it will be appreciated how other forms of further processing branches could be used to generate other types of classification results or other output types from a neural network. As an example, the neural network may be configured to perform a regression task, which could be used to generate (for instance) a score of a severity of a particular pathology from the medical image.

The combined processing branch 130 may comprise other layers suitable for a neural network, e.g. one or more additional convolution layers, normalization layers, fully connected layers and/or pooling layers. It is not essential for the combined processing branch to include a fully connected layer and/or activation function, as this can be omitted in some known examples of neural networks.

The neural network may be configured to provide a plurality of outputs. Reference to “an output” may refer to a single one of these outputs, or a combination of the outputs.

FIG. 2 illustrates another exemplary neural network 200 for use with embodiments.

The neural network 200 demonstrates another approach in which image data 101 is processed by an image processing branch 210 to generate image features 125. The non-image data elements 102 are combined (e.g. concatenated) with the image features 125 in a step 229 to generate input (features) for a combined processed branch 230. The combined processing branch 230 provides the output 150 of the neural network, which may be a classification, a score or any other suitable form of neural network output.

The combined processing branch may comprise layers suitable for a neural network, e.g. convolution layers and/or pooling layers. It is not essential for the combined processing branch 230 to include a fully connected layer or activation function, as this can be omitted in some known examples of neural networks, but these may be included in some other examples.

Optionally, the exemplary neural network 200 may include a non-image data element modification process 290, which may convert the non-image data elements into an image or feature map representation. This facilitates the use of conventional image processing techniques in the combined processing branch (e.g. pooling and/or convolutional layers or the like).

The non-image data element modification process may, for instance, convert time-varying parameters of the subject and/or medical imaging system (e.g. pulse rate, respiratory rate, imaging intensity or the like) into an image that represents the waveform of the time-varying parameter.

Embodiments of the present disclosure relate to approaches for identifying the influence of different parts of the medical image and the medical non-image data on the output of the neural network. This provides valuable information for assessing the main cause(s) of the output of the neural network, and therefore areas that would benefit from further investigation or attention from a clinician or operator.

Embodiments achieve this goal by quantifying the influence of each region (e.g. each pixel or group of pixels) of the medical image and non-image data element on the output of the neural network. This could be achieved by quantifying the influence of each region of the medical image and non-image data element on the output of the layer in the combined processing branch (as this has a direct influence/impact on the output). An indicator is then generated for each region and/or non-image data element, that indicates the quantified value of the influence of said region and/or non-image data element.

The indicator may contain a numeric measure, a categorical value and/or a binary value. Thus, the indicator may be a numeric indicator, a categorical indicator or a binary indicator.

One suitable example is described for use with the neural network illustrated by FIG. 1. In this example, the process of determining the quantified influence (i.e. the numeric value) for the regions of the medical image comprises generating, for each feature map or feature, a numeric measure of the influence of that feature map or feature on the output. As each feature map contains regions associated with a particular region of the original medical image, the influence of different regions or pixels of the medical image on the output can therefore be determined through appropriate identification of the influence of the regions of each feature map on the output.

One approach for identifying the influence of a non-image data element to the output of the neural network is to multiply the value of the non-image data element by the weight applied by the fully connected layer 121 to produce the output of the fully connected layer.

Thus, for an L-th non-image data element NI, the influence I_{L} of the non-image data element to the output of the fully connected layer may be equal to:

$${I}_{L} = {NI}_{L} . {w}_{L} \qquad ( 1 )$$

where w_{L} is equal to the weight applied to the L-th non-image data element NI.

One example for identifying the influence of different parts of the medical image makes use of a Class Activation Mapping (CAM) based technique. This approach assumes that the output of a neural network layer comprises one or more output values, and that input values (of the input) to the neural network are weighted and combined (e.g. summed or the like) to produce each output value, with weights being able to differ per output value.

Under this approach, for the medical image, the relevance of different regions of the medical image is considered at the level of the last convolutional layer 111 of the image processing branch 110. More specifically, the importance of a region in a feature map contributes to the importance of a corresponding region in the medical image.

A class activation map is used to represent the importance of regions of the medical image. Each feature map is multiplied by the weight applied to the image feature (derived from said feature map) to produce a preliminary class activation map or weighted feature map for each feature map. If there is more than one preliminary class activation map, the preliminary class activation map(s) are then combined (e.g. summed or multiplied) to produce the class activation map.

Assume that a k-th feature map FMₖ defines values FMₖ (i,j) for different regions or positions i, j, and wₖ is equal to the weight applied to the image feature derived from the k-th feature map by the fully connected layer 131. In this instance, a value M (i,j) at position i, j of the class activation map is calculated as:

$$M ( i , j ) = {\sum }_{k = 1}^{n} {FM}_{k} ( i , j ) \cdot {w}_{k} \qquad ( 2 )$$

A more complete description of performing a CAM based technique is disclosed by Zhou, Bolei, et al. “Learning deep features for discriminative localization.” Proceedings of the IEEE conference on computer vision and pattern recognition. 2016. The approach adopted by this disclosure may be used in embodiments of the present disclosure.

Other approaches for identifying the influence or importance of regions of a medical image and/or non-image data elements are hereafter described, and can be employed with any suitable neural network, such as those illustrated in FIG. 1 or 2.

One approach for identifying the influence or importance of regions of a medical image to an output of the neural network is disclosed by Selvaraju, Ramprasaath R., et al. “Grad-cam: Visual explanations from deep networks via gradient-based localization.” Proceedings of the IEEE international conference on computer vision. 2017. Embodiments of the present invention may make use of the approach adopted by this disclosure. This approach can be adapted for use with non-image data elements, e.g. when the non-image data element and the output of the neural network are both representable as a numeric value or values.

Another approach for identifying the influence or importance of a region of the medical image and/or non-image data element to the output of the neural network employs integrated gradients. An example approach is disclosed by Mukund Sundararajan, Ankur Taly, Qiqi Yan: Axiomatic Attribution for Deep Networks, 2017. This approach can be adapted for use with non-image data elements, e.g. when the non-image data element and the output of the neural network are both representable as a numeric value or values.

These two approaches thereby provide a gradient based mechanism for assessing the influence of a region of the medical image and/or a non-image data element on the output of the neural network. By way of working example, each non-image data element may be a respective value and the step of calculating, for each non-image data element, a numeric value may comprise, for each non-image data element: computing the gradient of the output with regard to the non-image data element; and calculating the product of the computed gradient and the non-image data element as the numeric value.

An approach that could be used to identify the influence or importance of a region of the medical image and/or a non-image data element is an Ablation CAM technique, such as that disclosed by Ramaswamy, Harish Guruprasad. “Ablation-cam: Visual explanations for deep convolutional network via gradient-free localization.” Proceedings of the IEEE/CVF Winter Conference on Applications of Computer Vision. 2020. This approach is to “zero”/“remove” individual feature channels and monitor the impact on the outcome. This approach can be adapted for use with non-image data elements, e.g. when the non-image data element and the output of the neural network are both representable as a numeric value or values. For instance, this approach could be used to identify the influence of a particular part of the input (e.g. a particular non-image data element) on the output of the neural network. For instance, a value of a non-image data element could be replaced with zero or a population mean, with the output being compared to the original output to identify an influence of the non-image data element.

As a working example, the output of the neural network and each non-image data element may be a respective value and the step of calculating, for each non-image data element, a numeric value may comprise, for each non-image data element: obtaining a first value of the output of the neural network when the non-image data element is omitted from first medical data input to the neural network; obtaining a second value of the output of the neural network when the non-image data element is included in second medical data input to the neural network, the second medical data being otherwise identical to the first medical data; defining a difference between the first value and the second value as a difference value; and calculating the product of the difference value and the non-image data element as the numeric value.

The skilled person would readily understand and implement various approaches for assessing the influence or importance of different image regions to the output of the fully connected layer and/or the output of the neural network.

Above-described embodiments make use of a neural network that produces a single output of the neural network. However, the skilled person would appreciate how a neural network may produces multiple outputs of the neural network (e.g. for different classes). Described approaches may be adapted for generating (for each region of the medical image or non-image data element) an indicator (of influence) for each class—i.e. each output of the neural network.

The skilled person will also appreciate how a neural network may output other forms of data, e.g. a score or measure. Of course, multiple such data elements may be output, and described approaches may be adapted for generating an indicator for each output data element.

Above-described neural networks are configured to process a medical image and corresponding non-medical data elements. The medical image may be a 2D or 3D image, and the term “pixel” is considered interchangeable with the term “voxel”. The medical image may form part of a sequence of medical images, e.g. a medical video. In some examples, the medical image may in fact comprise a set of separate medical images.

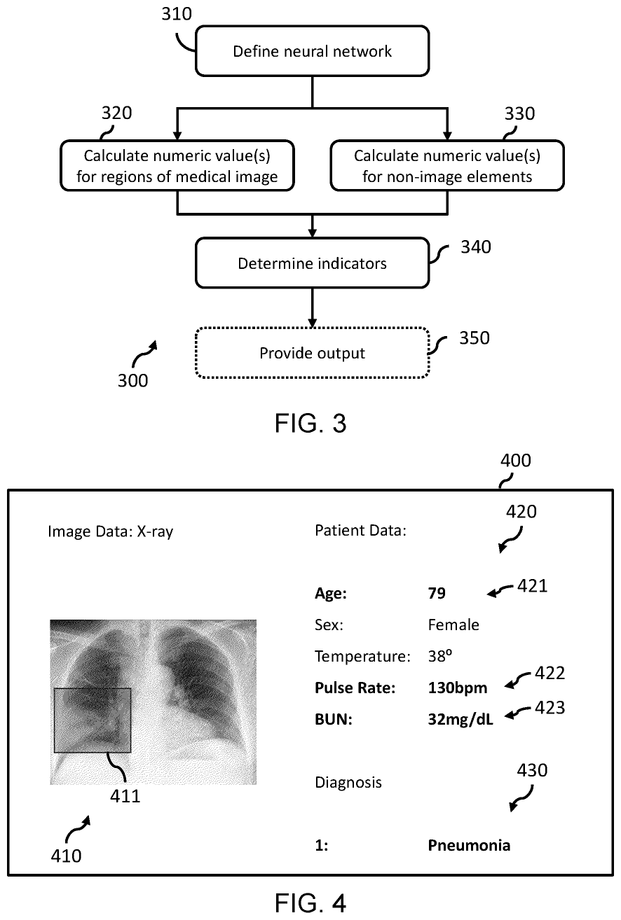

FIG. 3 illustrates a method 300 according to an embodiment.

The method determines the influence of inputs to a neural network on an output of the neural network.

The method 300 may comprise a step 310 of defining a neural network configured to process a medical image and one or more non-image data elements to generate an output, such as a classification, score, measure or other indicator. The neural network may be as previously described and is configured to: partially process the medical image using an image processing branch to derive one or more image features; combine the image features with the one or more non-image data elements to form intermediate inputs for a combined processing branch of the neural network; and process the intermediate inputs using the combined processing branch of the neural network to generate the output of the neural network.

The method further comprises a step 320 of calculating, for each of region of the medical image, a numeric value representing the influence of the region on the output of the neural network. Methods of calculating a numeric measure of the influence of each region have been previously described.

The method further comprises a step 330 of calculating, for each non-image data element, a numeric value representing the influence of the element on the output of the neural network. Method for performing step 330 have also been previously described.

The method 300 further comprises a step 340 of generating, for each region of the medical image and each and non-image data element, an indicator of the determined influence of the region or data element.

The indicator may be numeric indicator, i.e. contain a numeric value representing a predicted measure of influence of the region or data element on the output of the neural network. The numeric indicator may be on a predetermined scale (e.g. 0 to 1, 0 to 10, 1 to 10, 0 to 100 or 1 to 100).

As another example, the indicator may be a binary indicator, i.e. contain a binary value. The binary indicator/value indicates whether or not the numeric value (calculated in step 320, 330) exceeds a predetermined threshold. This may comprise comparing the numeric value of the indicator to the predetermined threshold.

As another example, the indicator may be a categorical indicator, i.e. contain a categorical value. In this example, step 340 may be performed by comparing the numeric value of the indicator to a plurality of predetermined thresholds (e.g. representing a boundary between different categories) and/or non-overlapping ranges. Different categories may represent different levels of influence, e.g. “Low”, “Medium” or “High”.

The method 300 may further comprise a step 350 of providing a user-perceptible output responsive to the indicator(s) generated in step 340.

Step 350 may comprise, for instance, providing a visual representation (e.g. at a display) of the medical image and/or the non-image data elements, and visually emphasizing any regions of the medical image and/or non-image data elements having an indicator that indicate the determined influence exceeds some predetermined value and/or meets some predetermined criteria. This provides a clinician with useful information for assessing the subject, and in particular, to understanding the possible causes and/or reasons for a predicted output of the neural network. A visual emphasisation may include use of different colors, transparencies, highlighting, circling, arrows, annotations and so on.

If binary indicators are generated, step 350 may be adapted to either visually emphasize or not visually emphasize a visual representation of the region and/or non-image data elements. If categorical indicators are generated, step 350 may be adapted to provide different visual emphasisation for different categories, e.g. using different colors. If numeric indicators are generated, step 350 may be adapted to provide different visual emphasisation for different numeric values (e.g. increasing values increases in intensity, opacity, value of a particular RGB color or group of RGB colors etc.).

Step 350 may be appropriately modified for any other form of user-perceptible output (e.g. an audio output). For instance, an audible alert or computer-generated voice may indicate any regions of the medical image or non-image data elements having indicators that meet some predetermined criteria.

The indicators are not only useful for improving a clinician's understanding, but may also prove useful in further processing for computer-aided analysis of the subject. For instance, image regions and/or non-image data elements (associated with indicators that meet some predetermined criteria) may be further processed to perform a further analysis task.

As one example, image regions and/or non-image data elements (associated with indicators that meet some predetermined criteria) may be stored in a database for later assessment.

As another example, only those image regions and/or non-image data elements associated with indicators that meet some predetermined criteria may be further processed using another machine-learning method, so that the inputs to the other machine-learning method are restricted. This other machine-learning method may be configured for performing another (e.g. more specific) processing task (e.g. classification task). This may provide a more directed processing of the most relevant parameters, to reduce extraneous and possibly deceptive data being input to the machine-learning method.

The method 300 may be performed alongside a method in which the neural network processes the medical image and the corresponding non-image data elements to produce the output of the neural network, e.g. a classification result.

FIG. 4 illustrates an example of a visual representation 400 provided by an embodiment of the invention, e.g. on a display, for the purposes of improved contextual understanding. In this example, the neural network is configured to generate a classification result for one or more pathologies, diseases and/or conditions of the subject.

The visual representation provides a visual representation of the medical image 410 (here: a Chest X-ray), a visual representation of the non-image data elements 420 (here: information about the patient) and a visual representation of the output of the neural network 430 (here: a diagnostic classification of pneumonia).

A region 411 of the medical image is visually emphasized (here: using a box and shading). This indicates that the emphasized region is highly influential on the output of the neural network (e.g. has numeric value representing influence that exceeds a predetermined value).

Similarly, some of the non-image data elements 421, 422, 423 are visually emphasized, here: using bold text emphasis, indicating that these elements are also highly influential on the output of the neural network (e.g. are associated with numeric values representing influence that exceed a predetermined value).

A clinician viewing the visual representation 400 would be able to readily identify the main causes of the output of the neural network, and use this information to assess an accuracy of the classification and/or direct their treatment towards symptoms that most heavily affect the output of the neural network (e.g. recommend a treatment to reduce pulse rate or blood urea nitrogen level). This provides useful information for assessing and treating the patient and their symptoms.

Embodiments make use of a neural network that processes input data (here: a medical image and non-image data elements) to produce an output such as a classification result. Example classification results include the identification of a particular structural element, abnormality, disease, condition, pathology or status of the anatomy represented by the medical image.

Approaches for training a neural network are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can repeated until the error converges, and the predicted output data entries are sufficiently similar (e.g. ±1%) to the training output data entries. This is commonly known as a supervised learning technique.

For example, where the machine-learning algorithm is formed from a neural network, (weightings of) the mathematical operation of each neuron may be modified until the error converges. Known methods of modifying a neural network include gradient descent, backpropagation algorithms and so on.

The training input data entries correspond to example medical images and corresponding non-image data elements. The training output data entries correspond to corresponding example desired output results (e.g. classifications or scores) of the medical images (e.g. produced by experts).

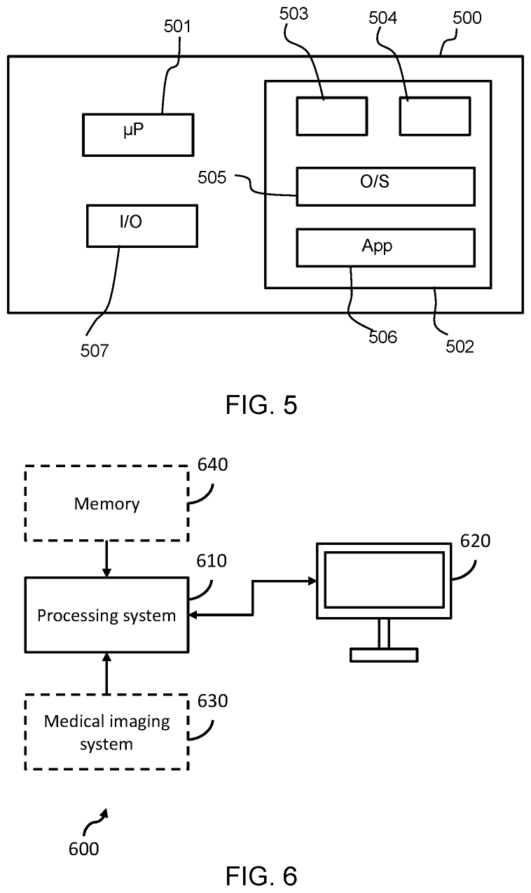

By way of further example, FIG. 5 illustrates an example of a processing system 500 within which one or more parts of an embodiment may be employed. Various operations discussed above may utilize the capabilities of the processing system 500. For example, one or more parts of a system for determining the influence of inputs to a neural network on an output generated by the neural network may be incorporated in any element, module, application, and/or component discussed herein. In this regard, it is to be understood that system functional blocks can run on a single computer or may be distributed over several computers and locations (e.g. connected via internet).

The processing system 500 includes, but is not limited to, PCs, workstations, laptops, PDAs, palm devices, servers, storages, and the like. Generally, in terms of hardware architecture, the processing system 500 may include one or more processors 501, memory 502, and one or more I/O devices 507 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 501 is a hardware device for executing software that can be stored in the memory 502. The processor 501 can be virtually any custom made or commercially available processor, a central processing unit (CPU), a digital signal processor (DSP), or an auxiliary processor among several processors associated with the processing system 500, and the processor 501 may be a semiconductor based microprocessor (in the form of a microchip) or a microprocessor.

The memory 502 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 502 may incorporate electronic, magnetic, optical, and/or other types of storage media. Note that the memory 502 can have a distributed architecture, where various components are situated remote from one another, but can be accessed by the processor 501.

The software in the memory 502 may include one or more separate programs, each of which comprises an ordered listing of executable instructions for implementing logical functions. The software in the memory 502 includes a suitable operating system (O/S) 505, compiler 504, source code 503, and one or more applications 506 in accordance with exemplary embodiments. As illustrated, the application 506 comprises numerous functional components for implementing the features and operations of the exemplary embodiments. The application 506 of the processing system 500 may represent various applications, computational units, logic, functional units, processes, operations, virtual entities, and/or modules in accordance with exemplary embodiments, but the application 506 is not meant to be a limitation.

The operating system 505 controls the execution of other computer programs, and provides scheduling, input-output control, file and data management, memory management, and communication control and related services. It is contemplated by the inventors that the application 506 for implementing exemplary embodiments may be applicable on all commercially available operating systems.

Application 506 may be a source program, executable program (object code), script, or any other entity comprising a set of instructions to be performed. When a source program, then the program is usually translated via a compiler (such as the compiler 504), assembler, interpreter, or the like, which may or may not be included within the memory 502, so as to operate properly in connection with the O/S 505. Furthermore, the application 506 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 507 may include input devices such as, for example but not limited to, a mouse, keyboard, scanner, microphone, camera, etc. Furthermore, the I/O devices 507 may also include output devices, for example but not limited to a printer, display, etc. Finally, the I/O devices 507 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 507 also include components for communicating over various networks, such as the Internet or intranet.

If the processing system 500 is a PC, workstation, intelligent device or the like, the software in the memory 502 may further include a basic input output system (BIOS) (omitted for simplicity). The BIOS is a set of essential software routines that initialize and test hardware at startup, start the O/S 505, and support the transfer of data among the hardware devices. The BIOS is stored in some type of read-only-memory, such as ROM, PROM, EPROM, EEPROM or the like, so that the BIOS can be executed when the processing system 500 is activated.

When the processing system 500 is in operation, the processor 501 is configured to execute software stored within the memory 502, to communicate data to and from the memory 502, and to generally control operations of the processing system 500 pursuant to the software. The application 506 and the O/S 505 are read, in whole or in part, by the processor 501, perhaps buffered within the processor 501, and then executed.

When the application 506 is implemented in software it should be noted that the application 506 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 506 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

FIG. 6 illustrates a system 600 according to an embodiment.

The system comprises a processing system 610 configured to perform any herein described method. In particular, the processing system may host a neural network for processing the medical image and non-image data elements to perform an analysis task, e.g. a classification, scoring, measuring or predictive task, as well as to generate indicators of the influence of different regions of the medical image and/or non-image data elements on (the output of) the neural network.

The processing system 610 may be configured as described with reference to FIG. 5.

The system 600 further comprises a user interface 620. The user interface 620 may be configured to provide a user-perceptible output (e.g. a visual representation) of any generated indicators. In some examples, the user interface 620 may provide a user-perceptible output of the medical image and/or non-image data elements

The user interface 620 may be further adapted to allow an operator to define the medical image (and thereby associated non-image data elements) that is processed by the neural network (hosted by the processing system 610).

The system 600 may further comprise a medical imaging system 630, configured to generate or obtain the medical image which is processed by the processing system 610. The medical imaging system may operate according to any known medical imaging modality, e.g.: X-ray imaging, CT imaging (a form of X-ray imaging), MR imaging, PET imaging, ultrasound imaging and so on.

The system 600 may further comprise a memory or storage unit 640, which may store medical images to be processed by the processing system 610 and/or the non-image data elements.

Thus, the processing system 610 may be configured to obtain the medical image(s) from the medical imaging system and/or memory unit 640, and the non-medical data element(s) from the memory storage unit 640 or the user interface 620.

It will be understood that disclosed methods are preferably computer-implemented methods. As such, there is also proposed the concept of a computer program comprising code means for implementing any described method when said program is run on a processing system, such as a computer. Thus, different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

1. A computer-implemented method of determining the influence of inputs to a neural network, that processes medical data, on an output of the neural network, the computer-implemented method comprising:
defining a neural network configured to process medical data to produce the output, the medical data comprising a medical image, comprising a plurality of different regions, and one or more non-image data elements, wherein the neural network is configured to:
partially process the medical image using an image processing branch, to derive one or more image features;
combine the image features with the one or more non-image data elements to form intermediate inputs for a combined processing branch of the neural network; and
process the intermediate inputs using the combined processing branch of the neural network to generate the output of the neural network;
calculating, for each of region of the medical image, a numeric value representing the influence of the region on the output of the neural network;
calculating, for each non-image data element, a numeric value representing the influence of the data element on the output of the neural network; and
determining, for each region of the medical image and each non-image data element, an indicator of the calculated numeric value representing the influence of the region of the medical image or the non-image data element.

2. The computer-implemented method of claim 1, further comprising a step of providing, at a user interface, a user-perceptible output responsive to each generated indicator.

3. The computer-implemented method of claim 1, wherein each indicator is a numeric indicator having a value equal to the calculated numeric value.

4. The computer-implemented method of claim 1, wherein the step of calculating, for each non-image data element, a numeric value comprises processing weights of the neural network to produce the numeric value representing the influence of the non-image data element.

5. The computer-implemented method of claim 1, wherein the combined processing branch comprises a fully connected layer that receives the intermediate inputs and an activation function that processes the output of the fully connected layer to generate the output of the neural network.

6. The computer-implemented method of claim 5, wherein:
the calculating, for each of region of the medical image, a numeric value comprises calculating a numeric value that represents the influence of the region on the output of the fully connected layer; and/or
the calculating, for each non-image data element, a numeric value comprises calculating a numeric value that represents the influence of the non-image data element on the output of the neural network.

7. The computer-implemented method of claim 6, wherein the step of calculating, for each non-image data element, a numeric value comprises, for each non-image data element:
identifying a weight applied to the non-image data element by the fully-connected layer of the neural network in producing one of the outputs of the fully-connected layer; and
calculating the product of the identified weight and the value of the non-image data element as the numeric value.

8. The computer-implemented method of claim 6, wherein:
the image processing branch comprises a penultimate layer that produces one or more feature maps, wherein regions of each feature map correspond to regions of the medical image, and a final layer that produces an image feature from each feature map; and
calculating, for each of region of the medical image, a numeric value comprises:
for each image feature:
identifying a weight applied to the image feature by the fully-connected layer to produce the output of the fully-connected layer; and
determining the product of the identified weight and the feature map, from which the image feature is derived, to produce a weighted feature map, and
determining, for each region of the medical image, a numeric value representing the influence of the region of the medical image based on the weighted feature maps.

9. The computer-implemented method of claim 8, wherein the step of determining, for each region of the medical image, a numeric value comprises:
summing the weighted feature maps to produce a class activation map, the class activation map contains numeric values representing the influence of different regions of the medical image on the output of the fully-connected layer and/or the output of the neural network.

10. The computer-implemented method of claim 1, wherein each non-image data element is a respective value and the step of calculating, for each non-image data element, a numeric value comprises, for each non-image data element:
computing the gradient of the output with regard to the non-image data element; and
calculating the product of the computed gradient and the non-image data element as the numeric value.

11. The computer-implemented method of claim 1, wherein the output of the neural network and each non-image data element is a respective value and the step of calculating, for each non-image data element, a numeric value comprises, for each non-image data element:
obtaining a first value of the output of the neural network when the non-image data element is omitted from first medical data input to the neural network;
obtaining a second value of the output of the neural network when the non-image data element is included in second medical data input to the neural network, the second medical data being otherwise identical to the first medical data;
defining a difference between the first value and the second value as a difference value; and
calculating the product of the difference value and the non-image data element as the numeric value.

12. The computer-implemented method of claim 1, wherein each indicator is a binary indicator that indicates whether or not the numeric value exceeds a predetermined threshold.

13. The computer-implemented method of claim 12, comprising:
providing, at a display, a visual representation of each non-image data element; and visually emphasizing the visual representations of any non-image data element associated with a binary value that indicates that the numeric value for the non-image data element exceeds the predetermined threshold; and/or
providing, at a display, a visual representation of the medical image; and visually emphasizing the visual representations of any region of the medical image associated with a binary value that indicates that the numeric value for the region of the medical image exceeds the predetermined threshold.

14. A computer program product comprising computer program code means which, when executed on a computing device having a processing system, cause the processing system to perform all of the steps of the method according to claim 1.

15. A processing system for determining the influence of inputs to a neural network, that processes medical data, on an output of the neural network, the processing system being configured to:
define a neural network configured to process medical data to produce the output, the medical data comprising a medical image, comprising a plurality of different regions, and one or more non-image data elements, wherein the neural network is configured to:
partially process the medical image using an image processing branch, to derive one or more image features;
combine the image features with the one or more non-image data elements to form intermediate inputs for a combined processing branch of the neural network; and
process the intermediate inputs using the combined processing branch of the neural network to generate the output of the neural network;
calculate, for each of region of the medical image, a numeric value representing the influence of the region on the output of the neural network;
calculate, for each non-image data element, a numeric value representing the influence of the data element on the output of the neural network; and
determine, for each region of the medical image and each non-image data element, an indicator of the calculated numeric value representing the influence of the region of the medical image or the non-image data element.
